(19) United States
(12) Patent Application Publication
(10) Pub. No.: US 2025/0288273 A1
(43) Pub. Date: Sep. 18, 2025

(54) METHOD AND DEVICE FOR REGULATING A DOSE WHEN RECORDING IMAGES OF AN OBJECT

(71) Applicant: Siemens Healthineers AG, Forchheim (DE)

(72) Inventor: Peter Horst SCHEUERING, Fuerth (DE)

(73) Assignee: Siemens Healthineers AG, Forchheim (DE)

(21) Appl. No.: 19/081,805

(22) Filed: Mar. 17, 2025

(30) Foreign Application Priority Data
Mar. 18, 2024 (DE) 10 2024 202 532.1

(51) Int. Cl. A61B 6/00; A61B 6/03; A61B 6/42

(52) U.S. Cl. CPC A61B 6/542; A61B 6/03; A61B 6/4208

(57) ABSTRACT

One or more example embodiments relates to a method for regulating a dose when recording images of an object via an image recording device having a radiation source and a pixel detector. One or more example embodiments further comprises a device, a control entity and an image recording system.

### CROSS-REFERENCE TO RELATED APPLICATION(S)

The present application claims priority under 35U.S.C. § 119 to German Patent Application No. 10 2024 202 532.1, filed Mar. 18, 2024, the entire contents of which are incorporated herein by reference.

### FIELD

One or more example embodiments relates to a method and a device for regulating a dose when recording images of an object, such as X-ray images, a control entity for controlling an image recording system and an image recording system, wherein the image recording system is preferably an X-ray system or a computed tomography system.

### RELATED ART

When images are recorded by exposing an object to radiation, the quality of a recorded image depends heavily on the strength of the radiation that struck the image detector during the time period of the recording. Therefore, X-ray recordings of adipose patients are darker than X-ray recordings of slim patients, for example, because a lower dose struck the detector due to absorption in the patent. However, images with a lower dose at the detector have a high level of noise, which is disadvantageous.

However, the image quality does not depend solely on the examined object, but also on the irradiation time. For example, if a plurality of recordings are made at short intervals one after the other, for example in the context of serial DER recordings (DFR: dark field radiography), only a short time can be used to irradiate the object for an individual image. This can also result in a lower dose (over the recording time) and therefore poor image quality.

In practice, when making serial DFR recordings, the length of time in which the radiation strikes the detector (the pulse duration) is limited by the image frequency and the readout time of the image detector. The power, i.e. essentially the beam intensity, is limited by the thermal load capacity of the emitter used and by the required total duration of the DER series.

For example, if a frequency of 10 images per second is specified, the pulse duration is limited to approximately 60 ms. If the total time of the DER series is 10 seconds, the power for a large focal point of 0.15 mm is limited to 50 kW. A voltage of 70 kV results in a current of 50 KW/70 kV, i.e. approximately 700 mA, which multiplied by the pulse time gives only 4.2 mAs. This current can be considered as a measure for the radiant flux. However, for good image quality with an object that is 20 cm thick, comparable radiography recordings require approximately 20 mAs, possibly 200 mAs for 30 cm, and even higher radiant flux in the case of thicker objects.

### SUMMARY

Until now, this problem has been addressed by increasing the acceleration voltage (in kV), which has an exponential effect on the X-ray energy. It is thereby possible to double the X-ray energy by increasing the voltage by only 15%, for example. However, increasing the acceleration voltage has the disadvantage that the images become harder and lose detail contrast.

A further means of increasing the image quality is to reduce the noise via noise suppression measures in the image processing. However, in addition to the noise, the noise suppression also eliminates important details in the image, which can present a problem during subsequent evaluation of the images.

Example embodiments specify a method and a device for regulating a dose when recording images of an object, a control entity for controlling an image recording system and an image recording system, via which the disadvantages described above can be avoided.

This object is achieved by a method according to claim 1, a device according to claim 10, a control entity according to claim 12 and an image recording system according to claim 13.

### BRIEF DESCRIPTION OF THE DRAWINGS

The invention is described in greater detail below on the basis of exemplary embodiments and with reference to the appended figures. Identical components in this case are denoted by identical reference numbers in the various figures, which are not generally to scale, and in which:

FIG. 1 shows a simple schematic illustration of a CT system with an exemplary embodiment of an inventive control entity for performing the method,

FIG. 2 shows a block diagram of the method sequence,

FIG. 3 shows the effects of one or more example embodiments on a data recording, and

FIG. 4 shows groupings in the case of a strip detector according to one or more example embodiments.

### DETAILED DESCRIPTION

A method according to one or more example embodiments is used for regulating a dose when recording images of an object. The recording is effected via an image recording device having a radiation source and a pixel detector. The method comprises the following steps:
exposing the object to radiation from the radiation source and recording an image using the pixel detector,
determining an irradiation value of the pixel detector, said irradiation value indicating a measure for the dose that is received by the detector, at least in a region of interest,
if the irradiation value lies outside a predetermined value range: changing the readout of the pixel detector with regard to a combination of adjacent pixels of the pixel detector into pixel groups, the pixels of a pixel group being read out jointly as a resulting pixel.

The method for regulating a dose is basically also a method for image recording, since it regulates how an image should be obtained from the detected signals.

The object can be in particular an (inorganic) item, a plant or a (human or animal) patient. The radiation that is used for recording can be electromagnetic radiation or particle radiation. For ease of understanding, the preferred case can be imagined in which the object is a person, of whom X-ray recordings (radiography or CT) are made.

A preferred detector can be a line detector or a detector which is resolved in two dimensions (comprising a multiplicity of image points in both an X-direction and a respectively orthogonal Y-direction), and is preferably a flat body detector. In this case, the word “pixel” is considered to have its original meaning of “picture element”, i.e. an image element, which can also mean that a pixel can have the form of a strip (in the case of a strip detector, for example).

Firstly, the object is exposed to radiation from the radiation source, whereby radiation strikes the pixel detector after passing through the object. There, the radiation generates free charge carriers which can be measured in each individual pixel or in combined pixel groups. A measurement is typically effected in such a way that the charge is converted by an ADC (analog-digital converter) into a digital value which indicates the strength of the charge.

If a pixel detector has been exposed to radiation, it is then also possible to determine an irradiation value (or irradiation flux). Said value is a measure for the dose that reached the detector. This can be done in various ways. The irradiation value is preferably determined for a predetermined region of the detector, which region may be the whole area or a partial area, for example the ROI (region of interest). The irradiation value can be calculated as a sum of the values of the pixels in the predetermined region, normalized if applicable, or from values that have been derived from the brightness values, for example dose values. An irradiation value is thus determined which indicates a measure for the dose received by the detector. The prior art essentially discloses how a measure for the dose at the detector can be obtained.

Once this irradiation value is available, a check then ascertains whether it lies outside a predetermined value range. In a simple case, this can preferably be effected by comparing the irradiation value with a limit value and, if this is not reached, it is assumed that too low a dose struck the detector. In a preferred, different and rather more complex case, the irradiation value can be compared with an upper and a lower limit value. If it exceeds the upper limit value, it is assumed that the detector received too high a dose (which could result in a reduction of the group size), and if the irradiation value does not reach the lower limit value, it is assumed that the detector received too low a dose (which could result in an increase of the group size). However, it is also preferably possible to perform a check using a plurality of predetermined value ranges, and ascertain the value range in which the irradiation value lies. A size of pixel groups can then be made dependent on the value range in which the irradiation value lies.

If the size of the pixel groups is to change, fewer or more mutually adjacent pixels than were previously available for the purpose of determining the irradiation value are combined into a (coherent) pixel group for the recording of the next image. A pixel group can also comprise a single pixel in this case.

A combination of pixels into a pixel group means that these pixels are read out jointly as a resulting pixel. This preferably means that the charge of all pixels in a pixel group is added (or taken together) and is only then converted into a value, for example by an ADC. This combining into pixel groups can also be referred to as “binning”.

In the case of a line detector, pixel groups are preferably formed from (linear) pixels which lie alongside each other. In the case of pixel detectors which are resolved in two dimensions, pixels are preferably combined into rectangular pixel groups having the width M pixels and the length N pixels, where M and N are natural numbers. M and N are preferably equal.

In the case of pixel groups comprising more than one pixel, pixels which lie alongside each other are therefore jointly binned and are consequently already consolidated in the detector when their signals are read out. For example, 4 pixels which lie in a square can be jointly binned and therefore read out together in the detector. This means that a significantly larger signal is already received by the ADC in the detector, and therefore both the X-ray noise and the amplifier noise of the ADC are reduced. Instead of 2×2 pixels, 9 pixels can also be combined into a 3×3 square as a pixel group.

As a result of automatically combining pixels into pixel groups, it is therefore possible effectively to suppress image noise if the dose at the detector is too low. The noise reduction achieved thereby is considerably better than noise reduction downstream of the ADC because the noise still occurs directly in the detector in the analog region, already before the signal is first processed.

In this case, the method can be employed in parallel with or as an alternative to a change of beam parameters (for example the acceleration voltage) or postprocessing of the images for the purpose of noise reduction. For example, serial recordings having a satisfactory image quality can therefore be performed using a comparatively low X-ray dose in the case of adipose patients.

As a result of the reduced resolution, the image admittedly loses detail sharpness but has significantly better contrast as a result of reducing the noise via the binning, so that the image which had to be recorded using too low a dose has a qualitatively better appearance using this method than via a higher kV and digital noise reduction. It is therefore also possible, for example, to perform examinations of very adipose objects using a higher image frequency and longer duration, such as are required in, for example, card angiography, with better image quality than previously. For it is thus possible to maintain the voltage of 73 kV, required for the iodine contrast method that is often used in angiography, for considerably longer.

A device according to one or more example embodiments is used to regulate a dose when recording images of an object via an image recording device which has a radiation source and a pixel detector. The device comprises the following components:
a determination unit which is designed to determine an irradiation value of the pixel detector, said irradiation value indicating a measure for the dose that is received by the detector, at least in a region of interest,
a checking unit which is designed to check whether an irradiation value lies outside a predetermined value range,
an adaptation unit which is designed to change the readout of the pixel detector with regard to a combination of adjacent pixels of the pixel detector into pixel groups, the pixels of a pixel group being read out jointly as a resulting pixel.

The function of the components of the device has been described above. The device is preferably designed to execute a method according to one or more example embodiments.

A control entity according to one or more example embodiments is used to control an image recording system, in particular an X-ray system or a computed tomography system. It comprises a device according to one or more example embodiments. Alternatively or additionally, it is designed to perform a method according to one or more example embodiments.

An image recording system according to one or more example embodiments is in particular an X-ray system or a computed tomography system. It comprises a control entity according to one or more example embodiments.

One or more example embodiments can be realized in particular in the form of a computer unit with suitable software. For this purpose, the computer unit can have, for example, one or more interworking microprocessors or similar. In particular, it can be realized in the form of suitable software program parts in the computer unit. A largely software-based realization has the advantage that previously used computer units can also be upgraded easily via a software or firmware update in order to operate in the inventive manner. In this respect, the object is also achieved by a corresponding computer program product with a computer program which can be loaded directly into a storage entity of a computer unit, with program modules for executing all steps of the inventive method when the program is executed in the computer unit. In addition to the computer program, such a computer program product can optionally comprise additional elements such as, for example, documentation and/or additional components, including hardware components, such as hardware keys (dongles, etc.) for using the software, for example.

For the purpose of transportation to the computer unit and/or storage on or in the computer unit, it is possible to use a computer-readable medium, for example a memory stick, a hard disk or other transportable or permanently installed data medium, on which are stored the program modules of the computer program which can be read in and executed by a computer unit.

Further particularly advantageous embodiments and developments of the invention are derived from the dependent claims and from the following description, it also being possible for the claims in one statutory class of claim to be developed in a similar manner to the claims and parts of the specification relating to another statutory class of claim and, in particular, for individual features of various exemplary embodiments or variants to be combined to form novel exemplary embodiments or variants.

In the event that the irradiation value indicates that too low a dose struck the pixel detector, in particular if the irradiation value lies below a predetermined lower limit value, pixels are preferably combined into pixel groups or, in the case of previously existing pixel groups, pixels are combined into larger pixel groups. Since individual pixels are considered to be the smallest possible pixel groups, in the event that the dose at the pixel detector was too low, larger pixel groups are formed than were available when determining the irradiation value.

In the event that the irradiation value indicates that too high a dose struck the pixel detector, in particular if the irradiation value lies above a predetermined upper limit value (upper limit value>lower limit value), existing pixel groups are rearranged into smaller pixel groups or resolved into individual pixels. Therefore if the dose at the pixel detector was too low, smaller pixel groups are formed, which can ultimately result in resolution into individual pixels.

A plurality of predefined value ranges are preferably provided, and binning is based on the value ranges within which the irradiation value lies. The lower the dose in a value range, the larger the chosen group.

The combination of pixels into pixel groups preferably takes place progressively. When using a detector that is resolved in two dimensions, in the case of a decreasing dose, 4 adjacent pixels are preferably combined in the form of a 2×2 group first, then 9 pixels in the form of a 3×3 group, and then 16 pixels in the form of a 4×4 group. Alternatively or additionally, in the case of an increasing dose, groups having a number of N×N pixels are made smaller, in particular by continuously reducing N by 1. When using a line detector, where N adjacent linear pixels form a pixel group, a pixel group is preferably formed by N+1 adjacent pixels in the case of a reducing dose, and by N−1 adjacent pixels in the case of an increasing dose.

The combination into groups ideally takes place automatically in this case. This can preferably take place on the basis of a predetermined hysteresis, where a smaller limit value applies for an increase in the number of pixels per group than for a decrease in the number of pixels per group.

In order that the individual images in a series have the same parameters, image regions depicted by groups are represented in a size that corresponds to the pixels of the group. A 2×2 group is represented as 2×2 pixels and a 3×3 group as 3×3 pixels. Therefore the resulting image still has the same pixel matrix size, even with higher binning rates.

According to a preferred embodiment variant of the method, provision is made for a regulating hierarchy which specifies a sequence for changing irradiation parameters and changing the readout of the pixel detector. In particular, it specifies when a combination of pixels into pixel groups takes place in a sequence with a change of the radiant flux and/or a change of the irradiation time period and/or a change of the acceleration voltage. In this case, the regulating hierarchy is preferably selected from a group of regulating hierarchies depending on the type of examination or the patient.

The noise in the images is usually acceptable as long as the value that is preferred by the user in respect of the acceleration voltage is satisfied and the regulation works using the radiant flux alone. However, as soon as this is no longer sufficient, there is an increase in both the noise value (due to too low a dose) and the hardening (due to a higher acceleration voltage). It is therefore possible to increase the group size first, before an optimal range of the acceleration voltage and/or the radiant flux is exceeded.

It should be noted here that the available beam parameters all have different effects on the image quality. The irradiation time has an effect on movement blurring. The longer the selected irradiation time, the more the image is blurred. The acceleration voltage has an effect on the image quality. The higher this is, the harder (i.e. poorer) the image. The radiation flux has an effect on the stability of the X-ray tubes, meaning that if this is increased instead of the acceleration voltage, it is possible for the tubes to become too hot already after a short time. The method for grouping can therefore be used selectively for a reduction of movement blurring, hardening or an increase in the stability, depending on what is most important to a user in the respective situation. However, this can also be derived automatically from the organ that is chosen to be examined. For the recording of neurons in the brain, for example, the stability would be important, whereas for the recording of a heart, which moves very quickly, the exposure should be kept very short in order to avoid any movement blurring.

It is therefore advantageous for a hierarchy, which determines when the group size should be changed in comparison with the irradiation time, the radiant flux and the acceleration voltage, to be dependent on the intended examination in each case.

A multiplicity of dose-regulated images are preferably recorded, particularly in the course of dark field radiography (a DER recording), tomosynthesis or a CT recording (computed tomography). The method is advantageous for a photon counting CT recording in particular. Provision is preferably made for determining the respective irradiation value for a plurality of these images and, at least for the subsequent recording, iteratively effecting an adaptation of the irradiation parameters and/or pixel groups.

The recording of a first image is preferably effected using a preset composition of pixel groups, for example with one pixel per pixel group, in order initially to record at the highest possible resolution. The radiation source in this case is so operated as to expose the object to radiation using preset radiation parameters, these specifying in particular a radiant flux, a duration of the emission of the radiation and/or an acceleration voltage. For example, it is possible to start with a normal acceleration voltage and a desired image repeat rate and a minimum radiant flux. However, it is also possible to consider which starting values would be optimal on the basis of the physique of the patient. An adapted composition of the pixel groups or alternatively adapted radiation parameters are then used if applicable for subsequent recordings of images of the same subject.

According to a preferred embodiment variant of the method, an irradiation value of the pixel detector is determined from a readout of the pixel detector, preferably by adding pixel values of a recorded image. In a simple case, the pixel values over the entire area of the image can simply be added and then represent the irradiation value. It is however the case that the edge regions of an X-ray image do not contain any information about the quality of the ROI (region of interest). It can therefore be advantageous to define an ROI beforehand and to derive the irradiation value solely from pixel values of the ROI. Since the ROI can vary from examination to examination, normalization is appropriate here for the purpose of comparison with limit values or value ranges, preferably a sum of the pixel values of the ROI divided by the image area of the ROI.

According to a preferred embodiment variant of the method, for a readout of pixels in a pixel group, the signals of said pixels are combined. In this case, the charges of the pixels in the corresponding semiconductor elements of the pixel detector are preferably added analogously. A digital value of the pixel group is only generated after said addition. This has the advantage of reducing the signal-to-noise ratio. Components for analog addition are disclosed in the prior art, for example analog adders from an operation amplifier. Such an addition can basically be applied in the case of all pixel detectors, including for example image amplifiers with add-on video camera.

It is also preferable to bin an image dynamically using different combinations. For example, in the event that the dose is sufficient in some regions and too low in other regions, the binning could be performed variously in different image regions (for example by combining a plurality of digital pixel values into groups and then normalizing). Thus, for example, the grouping in those regions in which the dose is sufficient can be selected differently than in regions in which the dose is too low (where the groups are made bigger). Concerning this, it must nonetheless be taken into consideration that, in the case of many detectors, individual measurements and calibrations will be required for different binnings. Adequate calibration should therefore be ensured. It is nonetheless advantageous in general for bright regions on the image detector to undergo less binning than darker regions.

In an embodiment variant of the method, it is preferable for each pixel group in an image to occupy the space of the pixels contained therein. The value of this “big” pixel relates to various possibilities which offer different advantages. In one preferred possibility, each of these pixels in a pixel group has the pixel value of the pixel group. Since this is composed of the analog sum of the pixel values, this alternative is very fast, but systematically depicts different brightnesses in the case of different group sizes. In a further preferred possibility, each of the pixels in a pixel group has a pixel value which corresponds to a normalized pixel value of the pixel group (for example, 3×3 group: pixel value/9). This admittedly requires a certain computing effort, but the brightnesses of different projection images are directly comparable, even with different group sizes. In a further preferred possibility, the pixel values of the pixels in a pixel group exhibit a progression between the pixel value of the pixel group and the pixel values of adjacent pixel groups. Such images have an artificially improved resolution, though this does not provide any additional medical information.

A preferred device is characterized in that the adaptation unit is designed to firstly combine pixel groups so that a combined signal of the pixel group is digitized, and/or to derive the size of pixel groups from a comparison by the checking unit with a plurality of limit values, and/or the device comprises a regulating unit in which various regulating hierarchies are stored and which selects a regulating hierarchy for changing a composition of pixel groups and/or radiation parameters. These functions have been described above.

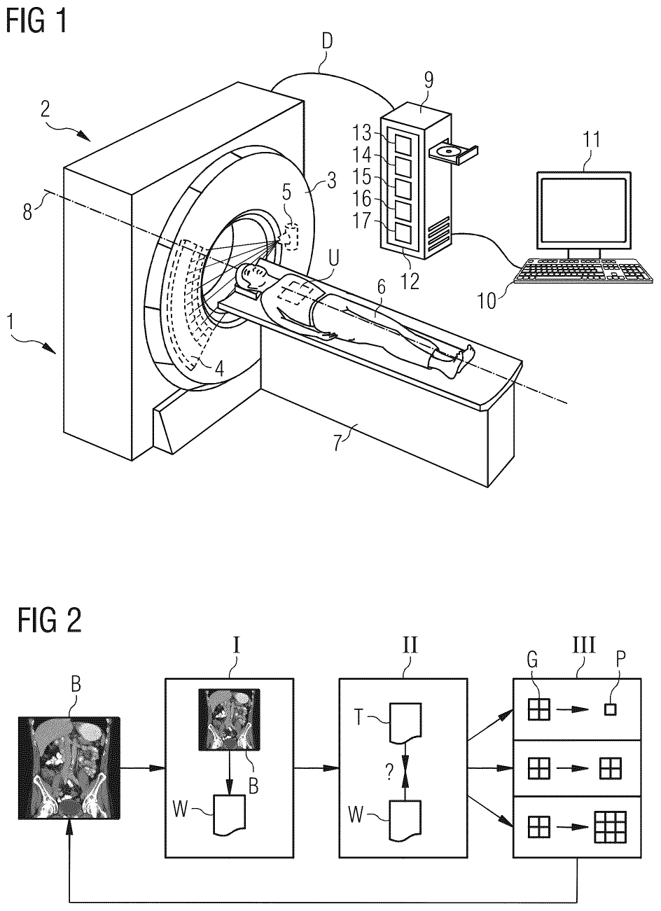

FIG. 1 shows an embodiment variant of a computed tomography system (CT system) 1, comprising a radiation detector 4 and a radiation source 5. The radiation source 5 is designed to expose the radiation detector 4 to radiation. The CT system 1 here comprises a gantry 2 with a rotor 3. The rotor 3 comprises an X-ray source 5 as the radiation source 5, and the radiation detector 4 which is designed to detect X-radiation.

The rotor 3 can be rotated about the axis of rotation 8. The patient 6 is supported on the patient couch 7 and can be moved along the axis of rotation 8 through the gantry 2. The head of the patient 6 rests on a support aid L. The computing unit 9 is provided for the purpose of controlling the imaging system 1 and/or generating an image data set based on the signals detected by the radiation detector 4.

A (raw) X-ray image data set of the examination object 6 is usually recorded from a plurality of angle directions via the radiation detector 4 using a beam energy in each case, i.e. two or more raw data sets. It is then possible to reconstruct a (final) image data set based on the (raw) X-ray image data set via a mathematical method, for example comprising a filtered back projection or an iterative reconstruction method.

The computing unit 9 is used here as a control entity 9 for controlling the CT system 1. Connected to this computing unit 9 is an input device 10 and an output device 11. The input device 10 and the output device 11 can, for example, allow interaction with the user or the representation of an image data set that is generated from the recorded pixel values D.

The control entity 9 comprises an inventive device 12 for regulating a dose when recording images of an object 6. The device 12 comprises a data interface 13, a determination unit 14, a checking unit 15, an adaptation unit 16 and a regulating unit 17.

The data interface 13 allows the device 12 to receive the required data, i.e. at least the image data (pixel values D) from the pixel detector 4 (radiation detector 4) and if applicable dose data from an additional measuring unit.

The determination unit 14 is used to determine an irradiation value W of the pixel detector 4, said irradiation value W indicating a measure for the dose that is received by the detector, at least in a region of interest. The checking unit 15 is used to check whether an irradiation value W lies outside a predetermined value range T. In this case, an irradiation value W can be derived directly from the pixel values D, i.e. essentially from the image brightness of the X-ray images B.

The adaptation unit 16 is used to change the readout of the pixel detector 4 with regard to a combination of adjacent pixels P of the pixel detector 4 into pixel groups G, the pixels P of a pixel group G being read out jointly as a resulting pixel P.

FIG. 2 shows a block diagram of the sequence of an inventive method for regulating a dose when recording images of an object 6.

In step I, the object 6 is exposed to X-radiation from the radiation source 5 and an image (specifically X-ray images B) recorded via the pixel detector 4. In this case, irradiation values W of the pixel detector 4 are determined, which indicate a measure for the dose that is received by the detector 4. An irradiation value W can be the dose value, for example, or simply the image brightness.

In step II, a check ascertains whether the irradiation value W lies outside a predetermined value range T.

If so, the readout of the pixel detector 4 is changed in step III with regard to a combination of adjacent pixels P of the pixel detector 4 into pixel groups G, the pixels P of a pixel group G being read out jointly as a resulting pixel P.

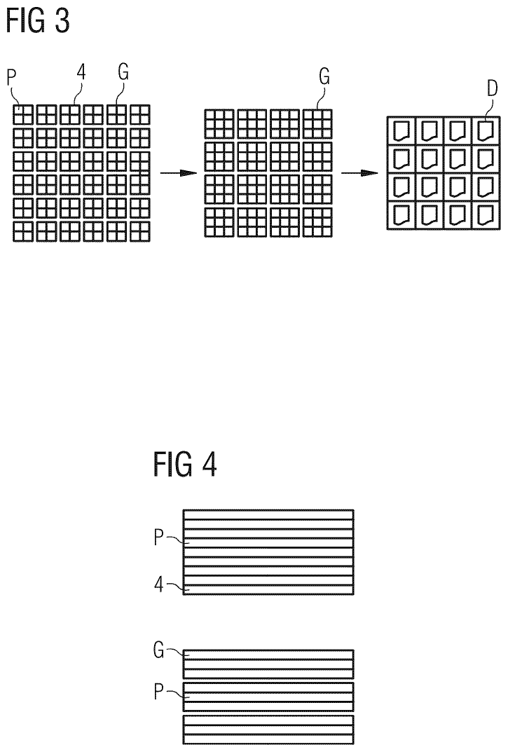

FIG. 3 shows the effects of the inventive method on a data recording. Firstly (left), pixels P of a radiation detector 4 are combined into 2×2 pixel groups G. In this case, the physical radiation detector 4 should be imagined without the intermediate spaces, which have only been inserted to illustrate the groupings. If it is now ascertained for a recording that, with such a grouping, the dose on the radiation detector 4 is too low, the size of the pixel groups G is automatically increased (center). In this case, the pixels P are combined to form 3×3 pixel groups G and are read out jointly. Therefore an image having 4×4 pixel values D is now obtained instead of an image previously having 6×6 pixel values D (or image values D).

It should be noted that in practice the radiation detectors have significantly more image points. In the case of images having a side width of 512×512 pixels P, a 2×2 grouping would still result in 256×256 pixels P and a 3×3 grouping in 170×170 pixels P.

FIG. 4 shows a potential grouping of pixels P of a strip detector into pixel groups G. The top of FIG. 4 shows the strip detector, whose pixels P extend over the entire width. The bottom of FIG. 4 shows a potential grouping of three pixels P into a pixel group G in each case. The detector looks physically the same as shown at the top, but three adjacent pixels P are always read out together by an ADC.

In conclusion, it is again noted that the invention described in detail above merely represents exemplary embodiments which can be modified in all manner of ways by a person skilled in the art without thereby departing from the scope of the invention. Furthermore, use of the indefinite article “a” or “an” does not preclude multiple instances of the features concerned. Likewise, terms such as “unit” do not preclude the relevant components consisting of multiple interacting sub-components, which can also be spatially distributed if applicable. The term “a number” is understood to signify “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors herein interpreted used accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, i steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, JavaR, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific

examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for regulating a dose when recording images of an object via an image recording device having a radiation source and a pixel detector, said method comprising:
exposing the object to X-radiation from the radiation source and recording an image via the pixel detector;
determining an irradiation value of the pixel detector, wherein the irradiation value indicates a measure for a dose received by at least a region of interest of the pixel detector;
checking whether the irradiation value is outside a predetermined value range; and
changing, based on the checking, a readout of the pixel detector with regard to a combination of adjacent pixels of the pixel detector into pixel groups, wherein the pixels of a single pixel group are read out jointly as a resulting pixel.

2. The method of claim 1, further comprising at least one of:
combining pixels into pixel groups or into larger pixel groups if the irradiation value lies below a predetermined lower limit value; or
rearranging existing pixel groups into smaller pixel groups or resolving existing pixel groups into individual pixels if the irradiation value exceeds a predetermined upper limit value.

3. The method of claim 2, wherein the combining takes place progressively.

4. The method of claim 1, wherein a regulating hierarchy is specified, the regulating hierarchy specifies a sequence for changing irradiation parameters and changing the readout of the pixel detector.

5. The method of claim 1, wherein a multiplicity of dose-regulated images are recorded.

6. The method of claim 1, wherein
the recording records a first image using a preset composition of pixel groups and the radiation source is operated as to expose the object to radiation using preset radiation parameters, the preset radiation parameters specifying at least one of a radiant flux, a duration of emission of the radiation or an acceleration voltage, and
at least one of an adapted composition of the pixel groups or adapted irradiation parameters are used for subsequent recordings of images of a same subject.

7. The method of claim 1, wherein the determining determines the irradiation value from a readout of the pixel detector by adding pixel values of a recorded image.

8. The method of claim 1, further comprising:
for a readout of pixels in a pixel group, combining signals of the pixels in the pixel group, wherein charges of the pixels in corresponding semiconductor elements of the pixel detector are added analogously, and a digital value of the pixel group is generated after the analog addition.

9. The method of claim 1, wherein
each pixel group in an image occupies a space of the pixels contained the pixel group,
each of these pixels in a particular pixel group has the pixel value of the particular pixel group, has a pixel value which corresponds to a normalized pixel value of the particular pixel group, or
the pixel values of the pixels in the particular pixel group exhibit a progression between the pixel value of the particular pixel group and the pixel values of adjacent pixel groups.

10. A device for regulating a dose when recording images of an object via an image recording device which has a radiation source and a pixel detector, the device comprising:
a determination unit configured to determine an irradiation value of the pixel detector, wherein the irradiation value indicates a value for a dose that is received at least in a region of interest by the pixel detector;
a checking unit configured to check whether the irradiation value is outside a predetermined value range; and
an adaptation unit configure to change a readout of the pixel detector with regard to a combination of adjacent pixels of the pixel detector into pixel groups, wherein the pixels of a single pixel group are read out jointly as a resulting pixel.

11. The device of claim 10, wherein at least one of,
the adaptation unit is configured to at least one of,
firstly combine pixel groups such that a combined signal of the pixel group is digitized, or
derive a size of pixel groups from a comparison by the checking unit with a plurality of limit values; or
the device comprises a regulating unit in which various regulating hierarchies are stored, the regulating unit configured to select a regulating hierarchy for changing at least one of a composition of the pixel groups or radiation parameters.

12. A control entity for controlling an image recording system comprising:
the device of claim 10.

13. An image recording system comprising:
the control entity of claim 12.

14. A computer program product, comprising instructions which when executed by a computer of a system, cause the system to perform the method of claim 1.

15. A non-transitory computer-readable storage medium, comprising instructions which when executed by a computer of a system, cause the system to perform the method of claim 1.

16. The method of claim 2, wherein a plurality of predefined value ranges is available and binning is based on the value ranges outside of which the irradiation value lies.

17. The method of claim 3, wherein in a case of a decreasing dose the combining includes:
first combining 4 adjacent pixels into a 2×2 group;
second combining 9 pixels into a 3×3 group; and
third combining 16 pixels into a 4×4 group.

18. The method of claim 3, wherein in a case of an increasing dose, the rearranging includes:
groups having a number of N×N pixels are made smaller by continuously reducing N by 1.

19. The method of claim 4, wherein the regulating hierarchy is selected from a group of regulating hierarchies depending on a type of examination or a patient.

20. The method of claim 5, wherein
the multiplicity of dose-regulated images are recorded in as part of a DER recording, a tomosynthesis or a CT recording,
the respective irradiation value is determined for a plurality of the dose-regulated images, and
an adaptation of at least one of the irradiation parameters or the pixel groups takes place iteratively at least for the subsequent recording.
